(19) United States
(12) Patent Application Publication
(10) Pub. No.: US 2008/0133276 A1
(43) Pub. Date: Jun. 5, 2008

(54) ARTIFICIAL BONE INFORMATION MANAGING SYSTEM

(75) Inventors: Keita ABE, Tokyo (JP); Go NAGATA, Chiba (JP); Hirokazu SATO, Tokyo (JP)

Correspondence Address: GREENBLUM & BERNSTEIN, P.L.C., 1950 ROLAND CLARKE PLACE, RESTON, VA, 20191, US

(73) Assignee: PENTAX CORPORATION, Tokyo (JP)

(21) Appl. No.: 11/947,852

(22) Filed: Nov. 30, 2007

(30) Foreign Application Priority Data
Nov. 30, 2006 (JP) 2006-324955

(51) Int. Cl. G06Q 10/00; G06F 17/30; G06F 17/40

(52) U.S. Cl. 705/3

(57) ABSTRACT

An artificial bone information managing system that performs the management of information on an artificial bone which is ordered to a designing and manufacturing institution by a medical institution and which is designed and manufactured on a user-by-user basis by the designing and manufacturing institution is provided. The system includes: an order information acquiring part that receives the order information of the artificial bone from a medical institution side terminal device and then stores the order information in an order information database; an order information output part that receives an extraction request of order information, extracts associated order information from the order information database and transmits the extracted order information to the designing and manufacturing institution side terminal device; an artificial bone information acquiring part that receives the artificial bone information from the designing and manufacturing institution side terminal device and stores the artificial bone information in an artificial bone information database; and an output processing part that receives an extraction request of artificial bone information, extracts associated artificial bone information from the artificial bone information database and transmits the extracted artificial bone information to the medical institution side terminal device or the user side terminal device, wherein all the information are transmitted and received through a network.

### BACKGROUND OF THE INVENTION

1. Field of the Invention

The present invention relates to an artificial bone information managing system.

2. Description of the Prior Art

There exists a custom-made artificial bone (customized artificial bone) manufactured on a user-by-user basis according to the situation of an individual patient (that is, a user) In the manufacture of the custom-made artificial bone, various diagnosis image data such as CT data of the user (patient) or a CT film is utilized. As one example of methods for manufacturing the artificial bone, there has been proposed an implant design method for designing an implant to be applied to a bone body having a bone lost portion, including: a step of producing three-dimensional data of the bone body based on a plurality of tomographic image data of the bone body; and a step of estimating a shape of the bone body supposed to be fitted in the bone lost portion by using shape data of a portion symmetrical to the bone lost portion of the three-dimensional data with respect to a substantially symmetrical plane of the three-dimensional data (see U.S. Pat. No. 7,113,841).

In the meantime, the diagnosis image data for use in the manufacture of the artificial bone are transported from a medical institution taking care of the patient as the user to an artificial bone manufacturing company (a designing and manufacturing institution) by virtue of manpower (e.g., of a sales person of the artificial bone manufacturing company). For this reason, the task of transporting the diagnosis image data is time-consuming, troublesome and costly. Further from the standpoint of personal information management, it is not desirable that a third party person carries the diagnosis image data of the user which must be managed by the medical institution. Furthermore, the user who undergoes surgery of the artificial bone and its family members are scarcely given an opportunity of, and a means for, clearly knowing information on the artificial bone surgically applied to the user prior to the surgical operation.

### SUMMARY OF THE INVENTION

It is an object of the present invention to provide an artificial bone information managing system capable of reducing the time, effort and cost involved in transporting the information needed to design a user-specifically manufactured artificial bone and also capable of efficiently offering the artificial bone information on a designed artificial bone.

In order to achieve the above-mentioned object, a first aspect of the present invention is directed to an artificial bone information managing system that performs the management of information on an artificial bone which is ordered to a designing and manufacturing institution by a medical institution and which is designed and manufactured on a user-by-user basis by the designing and manufacturing institution. The system comprising: an order information acquiring means that receives the order information including the diagnosis image data of the user required in designing the artificial bone from a medical institution side terminal device through a network and then stores the order information in an order information database; an order information output means that receives an extraction request of the order information through the network, extracts the associated order information from the order information database in response to the order information extraction request and transmits the extracted order information to the designing and manufacturing institution side terminal device through the network; an artificial bone information acquiring means that receives the artificial bone information on the artificial bone designed by the designing and manufacturing institution according to the order information from the designing and manufacturing institution side terminal device through the network and stores the artificial bone information in an artificial bone information database; and an output processing means that receives an extraction request of artificial bone information through the network, extracts the associated artificial bone information from the artificial bone information database in response to the artificial bone information extraction request and transmits the extracted artificial bone information to the medical institution side terminal device or the user side terminal device through the network.

According to the artificial bone information managing system of the present invention, it is possible to eliminate the need to transport diagnosis image data for use in the design (manufacture) of an artificial bone from a medical institution taking care of a patient as a user to an artificial bone designing and manufacturing institution by virtue of manpower (of a sales person of the designing and manufacturing company). Further, it is possible to save the time, effort and cost involved in transporting order information such as diagnosis image data of a patient, and it is also possible to remove any concern about the leakage of personal information which may occur when a third party person carries the diagnosis image data of the patient (personal user) which must be managed by the medical institution.

It is also possible to provide a doctor of the medical institution, the user who undergoes surgery of the artificial bone and its family members with an opportunity of, and a means for, minutely and clearly knowing information on the artificial bone surgically applied to the user prior to the surgical operation. This makes it possible to efficiently provide information on the artificial bone to the related persons.

In the artificial bone information managing system according to the first aspect of the present invention, it is preferred that the order information is deleted from the order information database after the order information has been transmitted to the designing and manufacturing institution side terminal device by means of the order information output means.

This makes it possible to reduce a risk that the order information is stolen from the order information database by an unauthorized third party person.

Further, in the artificial bone information managing system according to the first aspect of the present invention, it is preferred that the system further comprises an authentication processing means that transmits storage location addresses of the order information allotted in the order information database and authentication information specific to the designing and manufacturing institution required in authenticating the designing and manufacturing institution to the designing and manufacturing institution side terminal device before the order information output means receives the extraction request of the order information from the designing and manufacturing institution side terminal device, wherein the authentication processing means performs verification of the authentication information to thereby authenticate the designing and manufacturing institution.

This makes it possible to carry out a secure information management.

Further, in the artificial bone information managing system according to the first aspect of the present invention, it is also preferred that the system is configured so that at least one of the storage location addresses of the order information and the authentication information to be transmitted to the designing and manufacturing institution side terminal device is changed upon each transmission thereof.

This also makes it possible to carry out a secure information management more reliably.

Furthermore, in the artificial bone information managing system according to the first aspect of the present invention, it is also preferred that the order information database and the artificial bone information database are consolidated into a common one, and the storage location addresses of the order information are the same as the storage location addresses of the artificial bone information.

This makes it possible to carry out an information management easily.

A second aspect of the present invention is directed to an artificial bone information managing system that performs the management of information on an artificial bone which is ordered to a designing and manufacturing institution by a medical institution and which is designed and manufactured on a user-by-user basis by the designing and manufacturing institution. The artificial bone information managing system comprising: an order information acquiring means that receives the order information including the diagnosis image data of the user required in designing the artificial bone from a medical institution side terminal device through a network and then stores the order information in an order information database; an order information output means that receives an extraction request of the order information, extracts the associated order information from the order information database in response to the order information extraction request and outputs the extracted order information; an artificial bone information acquiring means that receives the artificial bone information on the artificial bone designed by the designing and manufacturing institution according to the order information in an artificial bone information database; and an output processing means that stores an extraction request of artificial bone information through the network, extracts the associated artificial bone information from the artificial bone information database in response to the artificial bone information extraction request and transmits the extracted artificial bone information to the medical institution side terminal device or the user side terminal device through the network, wherein all of the order information acquiring means, the order information output means, the artificial bone information acquiring means, and the output processing means are located at the side of the designing and manufacturing institution.

According to the artificial bone information managing system of the second aspect of the present invention, it is possible to save the time, effort and cost involved in transporting order information such as diagnosis image data of a patient, and it is also possible to remove any concern about the leakage of personal information which may occur when a third party person carries the diagnosis image data of the patient (personal user) which must be managed by the medical institution.

Further, it is also possible to provide a doctor of the medical institution, the user who undergoes surgery of the artificial bone and its family members with an opportunity of, and a means for, minutely and clearly knowing information on the artificial bone surgically applied to the user prior to the surgical operation. This makes it possible to efficiently provide information on the artificial bone to the related persons.

Furthermore, according to this system, the artificial bone information managing system can also serve as the designing and manufacturing institution side terminal device.

In the artificial bone information managing system according to the second aspect of the present invention, it is preferred that the system is configured so that the order information is deleted from the order information database after the order information has been transmitted by means of the order information output means.

This makes it possible to reduce a risk that the order information is stolen from the order information database by an unauthorized third party person.

Further, in the artificial bone information managing system according to the first or second aspect of the present invention, it is also preferred that the output processing means is configured so that when the artificial bone information extraction request from the medical institution side terminal device is received through the network, the output processing means extracts the associated artificial bone information from the artificial bone information database in response to the information extraction request and transmits the extracted artificial bone information to the medical institution side terminal device, and wherein the output processing means is also configured so that when the artificial bone information extraction request from the user side terminal device is received through the network, the output processing means extracts the associated artificial bone information from the artificial bone information database in response to the artificial bone information extraction request and transmits the extracted artificial bone information to the user side terminal device.

This makes it possible to more efficiently provide information on the artificial bone to the related persons.

Further, in the artificial bone information managing system according to the first or second aspect of the present invention, it is also preferred that the system further comprises an authentication processing means that transmits storage location addresses of the order information allotted in the order information database and authentication information specific to the medical institution required in authenticating the medical institution to the medical institution side terminal device before the order information acquiring means receives the order information from the medical institution side terminal device, wherein the authentication processing means performs verification of the authentication information to thereby authenticate the medical institution.

This makes it possible to carry out a secure information management.

Further, in the artificial bone information managing system according to the second aspect of the present invention, it is also preferred that the system is configured so that at least one of the storage location addresses of the order information and the authentication information to be transmitted to the medical institution side terminal device is changed upon each transmission thereof.

This also makes it possible to carry out a secure information management more reliably.

Furthermore, in the artificial bone information managing system according to the second aspect of the present invention, it is also preferred that the order information database and the artificial bone information database are consolidated into a common one, and the storage location addresses of the order information are the same as the storage location addresses of the artificial bone information.

This makes it possible to carry out an information management easily.

A third aspect of the present invention is directed to an artificial bone information managing system that performs the management of information on an artificial bone which is ordered to a designing and manufacturing institution by a medical institution and which is designed and manufactured on a user-by-user basis by the designing and manufacturing institution. The artificial bone information managing system comprises: an order information acquiring means that stores the order information including the diagnosis image data of the user required in designing the artificial bone in an order information database; an order information output means that receives an extraction request of the order information through the network, extracts the associated order information from the order information database in response to the order information extraction request and transmits the extracted order information to the designing and manufacturing institution side terminal device through the network; an artificial bone information acquiring means that receives the artificial bone information on the artificial bone designed by the designing and manufacturing institution according to the order information from the designing and manufacturing institution side terminal device through the network and stores the artificial bone information in an artificial bone information database; and an output processing means that receives an extraction request of artificial bone information through the network, extracts the associated artificial bone information from the artificial bone information database in response to the artificial bone information extraction request and transmits the extracted artificial bone information to the user side terminal device through the network, wherein all of the order information acquiring means, the order information output means, the artificial bone information acquiring means, and the output processing means are located at the side of the medical institution.

According to the artificial bone information managing system of the third aspect of the present invention, it is possible to save the time, effort and cost involved in transporting order information such as diagnosis image data of a patient, and it is also possible to remove any concern about the leakage of personal information which may occur when a third party person carries the diagnosis image data of the patient (personal user) which must be managed by the medical institution.

Further, it is also possible to provide a doctor of the medical institution, the user who undergoes surgery of the artificial bone and its family members with an opportunity of, and a means for, minutely and clearly knowing information on the artificial bone surgically applied to the user prior to the surgical operation. This makes it possible to efficiently provide information on the artificial bone.

Furthermore, the artificial bone information managing system can also serve as the medical institution side terminal device. This makes it possible for the medical institute to easily provide the information on the artificial bone to the user who undergoes surgery of the artificial bone and its family members prior to the surgical operation. As a result, the user and its family members can easily know the information on the artificial bone prior to the surgical operation.

In the artificial bone information managing system according to the third aspect of the present invention, it is preferred that the system is configured so that the order information is deleted from the order information database after the order information has been transmitted to the designing and manufacturing institution side terminal device by means of the order information output means.

This makes it possible to reduce a risk that the order information is stolen from the order information database by an unauthorized third party person.

Further, in the artificial bone information managing system according to the third aspect of the present invention, it is also preferred that the output processing means is configured so that when the artificial bone information extraction request from the user side terminal device is received through the network, the output processing means extracts the associated artificial bone information from the artificial bone information database in response to the artificial bone information extraction request and transmits the extracted artificial bone information to the user side terminal device through the network.

This makes it possible to more efficiently provide information on the artificial bone to the related persons.

Further, in the artificial bone information managing system according to the third aspect of the present invention, it is also preferred that the system further comprises an authentication processing means that transmits storage location addresses of the order information allotted in the order information database and authentication information specific to the designing and manufacturing institution required in authenticating the designing and manufacturing institution to the designing and manufacturing institution side terminal device before the order information acquiring means receives the order information from the designing and manufacturing institution side terminal device, wherein the authentication processing means performs verification of the authentication information to thereby authenticate the medical institution.

This also makes it possible to carry out a secure information management more reliably.

Further, in the artificial bone information managing system according to the third aspect of the present invention, it is also preferred that the system is configured so that at least one of the storage location addresses of the order information and the authentication information to be transmitted to the designing and manufacturing institution side terminal device is changed upon each transmission thereof.

This also makes it possible to carry out a secure information management more reliably.

Further, in the artificial bone information managing system according to the present invention, it is also preferred that the order information database and the artificial bone information database are consolidated into a common one, and the storage location addresses of the order information are the same as the storage location addresses of the artificial bone information.

This makes it possible to carry out a secure information management easily.

In the artificial bone information managing system of each of the first to third aspects of the present invention, it is preferred that the diagnosis image data of the user includes a plurality of tomographic image data which indicate an installation position of the artificial bone of the user.

This makes it possible to improve accuracy and efficiency in designing the artificial bone.

Further, in the artificial bone information managing system of each of the first to third aspects of the present invention, it is also preferred that the order information include one of the information on an electronic medical chart of the user and the information on a material of the artificial bone.

This makes it possible to obtain, without omission, the data required in designing the artificial bone, thereby assuring reliability and efficiency of the artificial bone design.

Furthermore, in the artificial bone information managing system of each of the first to third aspects of the present invention, it is also preferred that the artificial bone information includes at least one of the two-dimensional image data and three-dimensional image data of the designed artificial bone.

This makes it possible for the doctors of the medical institution or the user to easily and reliably recognize the designed artificial bone with a concrete and clear image.

Moreover, in the artificial bone information managing system of each of the first to third aspects of the present invention, it is also preferred that the artificial bone information includes at least one of a size, a material, a color, a function and a past use record of the designed artificial bone.

This also makes it possible for the doctors of the medical institution or the user to easily and reliably recognize the designed artificial bone with a concrete and clear image.

These and other objects, structures and results of the present invention will be apparent more clearly when the following detailed description of the preferred embodiments is considered taken in conjunction with the appended drawings.

### BRIEF DESCRIPTION OF THE DRAWINGS

FIG. 1 is a network configuration view (block diagram) showing a first embodiment of an artificial bone information managing system in accordance with the present invention.

FIG. 2 is a part of a flowchart illustrating an operation (an example of routine) of the artificial bone information managing system shown in FIG. 1.

FIG. 3 is a continued part of the flowchart shown in FIG. 2.

FIG. 4 is descriptions of the words to be contained in the respective blocks in the flowchart shown in FIGS. 2 and 3.

FIG. 5 is a network configuration view (block diagram) showing a second embodiment of the present artificial bone information managing system.

FIG. 6 is a network configuration view (block diagram) showing a third embodiment of the present artificial bone information managing system.

### DETAILED DESCRIPTION OF THE PREFERRED EMBODIMENTS

Hereinafter, an artificial bone information managing system (abbreviated as “ABIMS” in the figures) of the present invention will be described in detail with reference to preferred embodiments illustrated in the accompanying drawings.

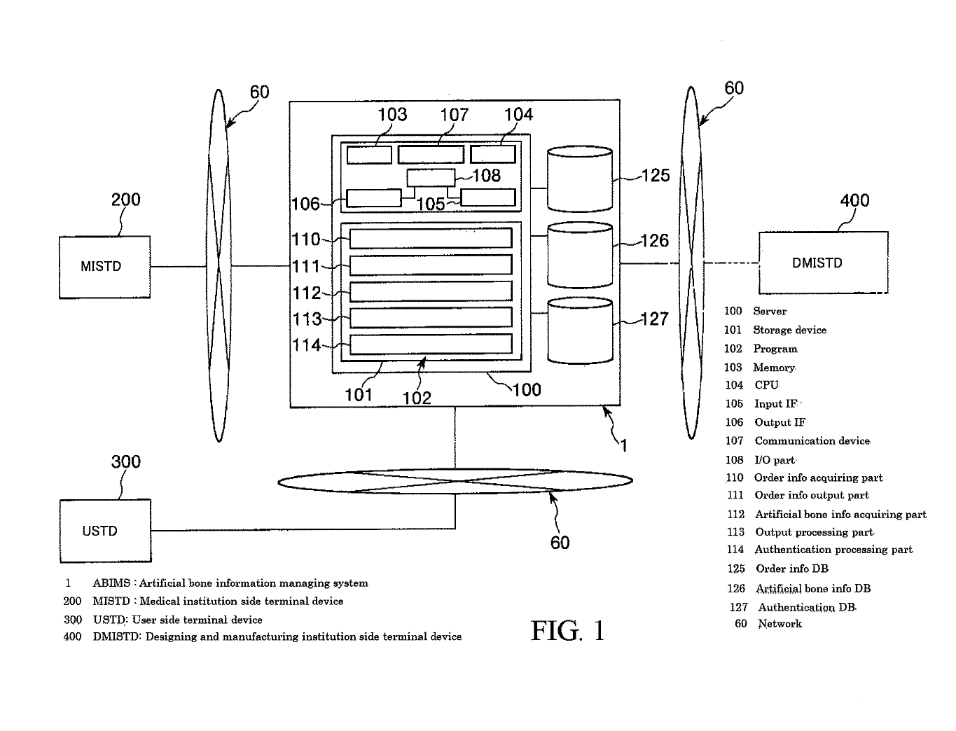

FIG. 1 is a network configuration view (block diagram) showing a first embodiment of an artificial bone information managing system in accordance with the present invention.

The artificial bone information managing system shown in FIG. 1 is a system that performs the management of information on an artificial bone (custom-made artificial bone) which is ordered to a designing and manufacturing institution by a medical institution and which is designed and manufactured on a user-by-user (patient-by-patient) basis by the designing and manufacturing institution.

In the present embodiment, it is assumed that the artificial bone information managing system 1 exists between a terminal device of the medical institution (a medical institution side terminal device or computer) (abbreviated as “MISTD” in the figures) 200 and a terminal device of the designing and manufacturing institution (a designing and manufacturing institution side terminal device or computer) (abbreviated as “DMSTD” in the figures) 400 and between a terminal device of the user as a patient under a care of the medical institution (a user side terminal device or computer) (abbreviated as “USTD” in the figures) 300 and the designing and manufacturing institution side terminal device 400, wherein the artificial bone information managing system 1 mediates between the terminal devices 200 and 400 and between the terminal devices 300 and 400, and manages information such as order information and artificial bone information. The number of each of the medical institution, the user as a patient under a care of the medical institution and the designing and manufacturing institution may be either single or plural. In the illustrated embodiment, each of the medical institution, the user as a patient under a care of the medical institution and the designing and manufacturing institution is shown in a single number for the simplicity of description.

The designing and manufacturing institution may be either an institution that designs an artificial bone (e.g., an implant) and manufactures the artificial bone based on the design (design information) or an institution that performs only the design of the artificial bone. In the latter case, the manufacture of the artificial bone is performed by other institutions based on the design of artificial bone prepared by the designing and manufacturing institution. The other institutions may be, e.g., the medical institution that orders the artificial bone, in which case the design information (design data) on the artificial bone is transmitted from the designing and manufacturing institution to the medical institution and then the medical institution manufactures the artificial bone based on the design information.

As shown in FIG. 1, the artificial bone information managing system 1 includes a server (computer) 100 which in turn includes a storage device (storage means) 101, a memory (a memory part) 103 and a CPU (control means) 104. The server 100 is adapted to manage various kinds of information in the artificial bone information managing system 1. A specified program 102 is stored in the storage device (storage means) 101. The program 102 is retrieved into the memory (memory part) 103 and executed by the CPU (control means) 104.

The server 100 of the artificial bone information managing system 1 includes an input interface (input IF) 105, an output interface (output IF) 106, a communication device 107 that governs communication (data exchange) with the external devices such as the medical institution side terminal device 200, the user side terminal device 300 and the designing and manufacturing institution side terminal device 400, and an I/O part 108. The server 100 is connected through a network 60 to the external devices such as the medical institution side terminal device 200, the user side terminal device 300 and the designing and manufacturing institution side terminal device 400 to perform data exchange therewith. The I/O part 108 is provided between the respective function parts and the communication device 107 of the artificial bone information managing system 1 to perform data buffering and various kinds of mediation processing.

The network 60 may be either an open network or a closed network. Examples of the network 60 include the Internet and a LAN, but the Internet is more preferred. It is also possible to use plural kinds of networks in combination.

The artificial bone information managing system 1 further includes an order information database 125 that stores order information (order data) including diagnosis image data of the user required in designing the artificial bone, an artificial bone information database 126 that stores artificial bone information (artificial bone data) on the artificial bone designed by the designing and manufacturing institution according to the order information, and an authentication database 127 that stores authentication information (authentication data) used in authentication processing.

Next, description will be made on the respective function parts that are configured and maintained by the artificial bone information managing system 1 according to the program 102.

The artificial bone information managing system 1 includes an order information acquiring part (order information acquiring means) 110 that receives the order information including the diagnosis image data of the user required in designing the artificial bone from the medical institution side terminal device 200 through the network 60 and then stores the order information in the order information database 125.

The diagnosis image data are not particularly limited as long as they can be used in designing the artificial bone. Examples of the diagnosis image data include a plurality of tomographic image data, such as CT data (data outputted from a CT device), which indicate an installation position of the artificial bone of the user, and measurement data of an MRI (Magnetic Resonance Imaging) device. Among these data, the plurality of tomographic image data indicating an installation position of the artificial bone of the user is preferred. This makes it possible to improve accuracy and efficiency in designing the artificial bone.

It is preferable for the order information to include one of the information on an electronic medical chart of the user and the information on a material of the artificial bone. More preferably, the order information includes both of the information. This makes it possible to obtain, without omission, the data required in designing the artificial bone, thereby assuring reliability and efficiency of the artificial bone design.

The artificial bone information managing system 1 further includes an order information output part (order information output means) 111 that receives an extraction request of order information through the network 60, extracts the associated order information from the order information database 125 in response to the order information extraction request and transmits the extracted order information to the designing and manufacturing institution side terminal device 400 through the network 60. The order information is deleted from the order information database 125 after it has been transmitted to the designing and manufacturing institution side terminal device 400 by means of the order information output part 111. This makes it possible to reduce a risk that the order information is stolen from the order information database 125 by an unauthorized third party person.

The artificial bone information managing system 1 further includes an artificial bone information acquiring part (artificial bone information acquiring means) 112 that receives the artificial bone information on the artificial bone designed by the designing and manufacturing institution according to the order information from the designing and manufacturing institution side terminal device 400 through the network 60 and stores the artificial bone information in the artificial bone information database 126.

Examples of the artificial bone information include two-dimensional image data, three-dimensional image data, a size, a material (e.g., appetite), a color, a function (e.g., a function of complementing a lost portion) and a past use record (e.g., a breakage rate and a contamination rate) of a designed artificial bone, among which the two-dimensional image data or the three-dimensional image data are preferred.

In other words, it is preferable for the artificial bone information to include one of the two-dimensional image data or the three-dimensional image data of the designed artificial bone. More preferably, the artificial bone information includes both of these data. This makes it possible for the doctors of the medical institution or the user to easily and reliably recognize the designed artificial bone with a concrete and clear image.

Furthermore, it is preferable for the artificial bone information to include at least one of the size, material, color, function and past use record of the designed artificial bone.

The artificial bone information managing system 1 further includes an output processing part (output processing means) that receives an extraction request of artificial bone information through the network 60, extracts the associated artificial bone information from the artificial bone information database 126 in response to the artificial bone information extraction request and transmits the extracted artificial bone information to the medical institution side terminal device 200 or the user side terminal device 300 through the network 60.

In a case where the extraction request of the artificial bone information is received from the medical institution side terminal device 200 through the network 60, the output processing part 113 extracts the associated artificial bone information from the artificial bone information database 126 in response to the extraction request and transmits the extracted artificial bone information to the medical institution side terminal device 200 through the network 60. In a case where the extraction request of the artificial bone information is received from the user side terminal device 300 through the network 60, the output processing part 113 extracts the associated artificial bone information from the artificial bone information database 126 in response to the extraction request and transmits the extracted artificial bone information to the user side terminal device 300 through the network 60.

The medical institution side terminal device 200 stores the received artificial bone information in a storage device (storage means) not shown in the drawings. Then, the artificial bone information is outputted to various kinds of external devices such as a display (display means) and a printer. That is to say, the artificial bone information is displayed on the display as an electronic image and also printed on a sheet of print paper so that the doctors of the medical institution can see the artificial bone information.

The user side terminal device 300 stores the received artificial bone information in a storage device (storage means) not shown in the drawings. Then, the artificial bone information is outputted to various kinds of external devices such as a display (display means) and a printer. That is to say, the artificial bone information is displayed on the display as an electronic image and also printed on a sheet of print paper so that the user or its family members can also see the artificial bone information. By doing so, the artificial bone information can be provided in an efficient manner.

In this regard, the artificial bone information managing system 1 further includes an authentication processing part (authentication processing means) 114 that transmits storage location addresses of the order information allotted in the order information database 125 and authentication information (e.g., an ID and a password) specific to the medical institution required in authenticating the medical institution to the medical institution side terminal device 200 before the order information acquiring part 110 receives the order information from the medical institution side terminal device 200, and also transmits storage location addresses of the order information allotted in the order information database 125 and authentication information (e.g., an ID and a password) specific to the designing and manufacturing institution required in authenticating the designing and manufacturing institution to the designing and manufacturing institution side terminal device 400 prior to receiving the extraction request of the order information from the designing and manufacturing institution side terminal device 400.

The authentication processing part 114 performs verification of the authentication information based on the authentication data stored in the authentication database 127 to thereby authenticate the medical institution and the designing and manufacturing institution. This makes it possible to prevent an unauthorized third party person from illegally using the information.

In this case, it is preferable to change one of the storage location addresses of the order information and the authentication information transmitted to the medical institution side terminal device 200 upon each transmission thereof. More preferably, both of the storage location addresses of the order information and the authentication information are changed upon each transmission thereof. Similarly, it is preferable to change one of the storage location addresses of the order information and the authentication information transmitted to the designing and manufacturing institution side terminal device 400 upon each transmission thereof. More preferably, both of the storage location addresses of the order information and the authentication information are changed upon each transmission thereof.

Although the order information database 125 and the artificial bone information database 126 may be separate from each other, it is preferred that they are consolidated into a common one. It is also preferred that the storage location addresses of the order information are the same as the storage location addresses of the artificial bone information.

The respective function parts (functions) of the artificial bone information managing system 1, including the order information acquiring part 110, the order information output part 111, the artificial bone information acquiring part 112, the output processing part 113 and the authentication processing part 114, may be realized either by hardware (not shown) or by a program (software) stored in the specified storage device 101 such as a memory or an HDD (Hard Disk Drive) as in the present embodiment. In the latter case, the CPU 104 of the artificial bone information managing system 1 retrieves a relevant program into the memory 103 and then executes the program.

Next, description will be made on configuration examples of the order information database 125, the artificial bone information database 126 and the authentication database 127 employed in the present embodiment.

The order information database 125 is a database for storing the order information of the artificial bone received from the medical institution side terminal device 200 through the network 60. In this order information database 125, the information is stored, for example, in a state that the storage location address of each of the order information is associated with the medical institution name, the user (patient) name, the order information (e.g., diagnosis image data) and so forth.

The artificial bone information database 126 is a database for storing the artificial bone information on the artificial bone designed by the designing and manufacturing institution side terminal device 400, which is received from the designing and manufacturing institution side terminal device 400 through the network 60. In this artificial bone information database 126, the information is stored, for example, in a state that the storage location address of each of the artificial bone information (which is preferably the same as the storage location address of the order information as set forth above) is associated with the medical institution name, the user name, the artificial bone information (e.g., two-dimensional or three-dimensional image data) and so forth.

The authentication database 127 is a database for storing the authentication information (authentication data) used in the authentication processing performed at the time of reception and use of the respective information. In this authentication database 127, the information is stored, for example, in a state that each authentication ID is associated with the medical institution name, the authentication password and the like in the case of the medical institution, the authentication ID is associated with the user name, the authentication password and the like in the case of the user, and the authentication ID is associated with the designing and manufacturing institution name, the authentication password and the like in the case of the designing and manufacturing institution.

The user is notified by the medical institution of the storage location addresses of the authentication information (e.g., the authentication ID and the password) and other information held by the medical institution and is able to use the authentication information.

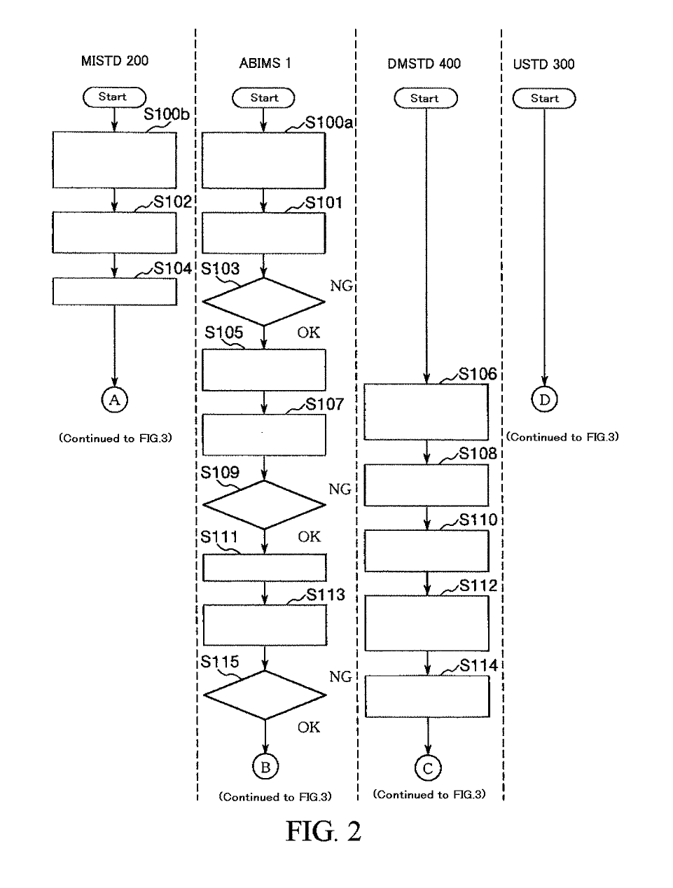

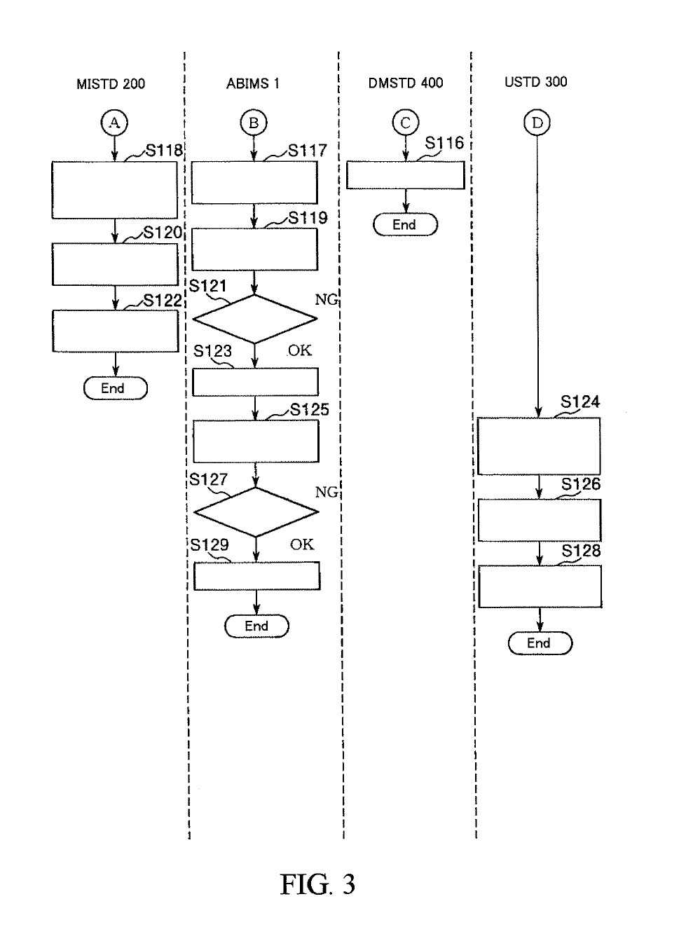

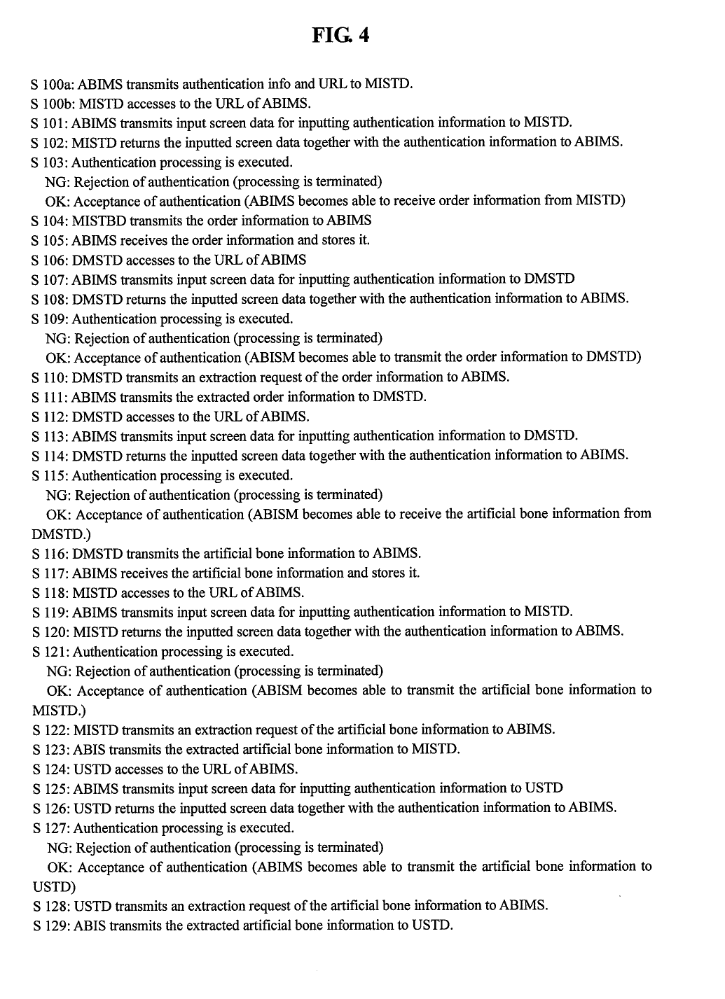

Next, an operation of the artificial bone information managing system, i.e., an example of routine of the artificial bone information management, will be described with reference to the flowchart shown in FIGS. 2 and 3. Note that FIG. 4 is descriptions of the words to be contained in the respective blocks in the flowchart shown in FIGS. 2 and 3.

In this regard, description will be made by way of example under the assumption that the artificial bone information managing system 1 exists between the medical institution side terminal device 200 and the designing and manufacturing institution side terminal device 400 and between the user side terminal device 300 and the designing and manufacturing institution side terminal device 400, wherein the artificial bone information managing system 1 mediates between the terminal devices 200 and 400 and between the terminal devices 300 and 400, and manages various kinds of information such as the order information and the artificial bone information. It is also assumed that the designing and manufacturing institution manages the artificial bone information managing system 1. In other words, it is assumed that a person-in-charge of the designing and manufacturing institution is aware of not only an authentication ID, a password and a URL of the medical institution but also an authentication ID and a password of the designing and manufacturing institution, which are to be transmitted to the medical institution side terminal device 200.

FIGS. 2 and 3 are flowcharts illustrating an operation (an example of routine) of the artificial bone information managing system shown in FIG. 1.

The respective operations of the artificial bone information managing system described below are realized by the program retrieved from the storage device 101 into the memory 103 and executed by the artificial bone information managing system 1.

An artificial bone is used in a surgical operation (a bone-lost-portion reconstructing method) by which an implant (the artificial bone) made of an artificial material is embedded in and fixed to a bone lost portion in case a cranial bone (including a cheek bone and a jaw bone) is partially lost by, e.g., a traffic accident or a brain tumor excision. In this case, a patient to whom the artificial bone is surgically applied is the user and the medical institution (doctors in charge) that takes care of the user issues an order of the artificial bone.

The doctors of the medical institution issuing an order of manufacture of the artificial bone makes a request for manufacture of the artificial bone by gaining access to the artificial bone information managing system 1 through the medical institution side terminal device 200 (not shown) or by contacting the designing and manufacturing institution through other communication means such as a telephone.

First, as shown in FIG. 2, the authentication processing part 114 of the artificial bone information managing system 1 transmits an authentication ID, a password and a URL (Uniform Resource Locator) of the Internet (storage location addresses of the order information and the artificial bone information) to the medical institution side terminal device 200 through the network 60 (step S100a). The expression “through the network 60” will be omitted in the following description.

In this connection, the doctors notify the user or its family members of the authentication ID, the password and the URL so that the user or its family members can use the authentication ID and the password.

The doctors gain access to the URL of the artificial bone information managing system 1 through the medical institution side terminal device 200 (step S100b).

Next, the authentication processing part 114 of the artificial bone information managing system 1 transmits input screen data for inputting the authentication ID and the password and the like (authentication information) to the medical institution side terminal device 200 (step S101).

Subsequently, the medical institution side terminal device 200 receives and screen-inputs the input screen data. Upon reception of the authentication ID and the password inputted by the doctors, the medical institution side terminal device 200 returns the received information back to the artificial bone information managing system 1 (step S102). The artificial bone information managing system 1 receives the returned information and the authentication processing part 114 thereof executes the authentication processing of the medical institution side terminal device 200 using the authentication database 127 (step S103).

If the result of the authentication processing shows rejection of authentication (authentication NG in step S103), the processing is terminated. In contrast, if the result of the authentication processing reveals acceptance of authentication (authentication OK in step S103), the order information acquiring part 110 of the artificial bone information managing system 1 becomes able to receive the order information from the medical institution side terminal device 200.

The medical institution side terminal device 200 is operated by the doctors to transmit the order information of the artificial bone (the diagnosis image data of the user, etc.) to the artificial bone information managing system 1 (step S104).

The order information acquiring part 110 of the artificial bone information managing system 1 receives the order information transmitted from the medical institution side terminal device 200 and then stores the order information in the associated storage location addresses of the order information database 125 (step S105).

At the time when the medical institution side terminal device 200 transmits the order information to the artificial bone information managing system 1, a password may be assigned to the order information to thereby assure enhanced security. If necessary, the data of the order information may be encrypted or compressed. In these cases, the doctors of the medical institution notify the designing and manufacturing institution of the password or the like. In the designing and manufacturing institution side terminal device 400, the password or the like thus notified is used in receiving the order information from the artificial bone information managing system 1.

Thus, the order information transmitted from the medical institution side terminal device 200 is stored in the order information database 125. A notice of completion of storage of the order information is transmitted from the artificial bone information managing system 1 to the designing and manufacturing institution side terminal device 400. The person-in-charge of the designing and manufacturing institution can understand from this notice that the storage of the order information has been completed.

The person-in-charge of the designing and manufacturing institution gains access to the URL of the artificial bone information managing system 1 through the designing and manufacturing institution side terminal device 400 (step S106).

Next, the authentication processing part 114 of the artificial bone information managing system 1 transmits input screen data for inputting the authentication ID and the password and the like (authentication information) to the designing and manufacturing institution side terminal device 400 (step S107).

Then, the designing and manufacturing institution side terminal device 400 receives and screen-inputs the input screen data. Upon reception of the authentication ID and the password inputted by the person-in-charge, the designing and manufacturing institution side terminal device 400 returns the received information back to the artificial bone information managing system 1 (step S108). The artificial bone information managing system 1 receives the returned information and the authentication processing part 114 thereof executes the authentication processing of the designing and manufacturing institution side terminal device 400 using the authentication database 127 (step S109).

If the result of the authentication processing shows rejection of authentication (authentication NG in step S109), the processing is terminated. In contrast, if the result of the authentication processing reveals acceptance of authentication (authentication OK in step S109), the order information acquiring part 110 of the artificial bone information managing system 1 becomes able to transmit the order information to the designing and manufacturing institution side terminal device 400.

The designing and manufacturing institution side terminal device 400 is operated by the person-in-charge to transmit an extraction request of the order information to the artificial bone information managing system 1 (step S110).

Responsive to the extraction request, the order information output part 111 of the artificial bone information managing system 1 extracts the associated order information from the storage location addresses of the order information database 125 and transmits the extracted order information to the designing and manufacturing institution side terminal device 400 (step S111).

The designing and manufacturing institution acquires the order information and designs an artificial bone based on the acquired order information. In a case where the artificial bone is used in, e.g., the bone-lost-portion reconstructing method as mentioned above, three-dimensional image data are produced from the diagnosis image data of the user in designing the artificial bone. Based on the three-dimensional image data, lost bone body shape data are produced by estimating the shape of a bone body (lost bone body) which is supposed to exist in the bone lost portion before the bone is lost.

Also produced is the artificial bone information on the designed artificial bone (two-dimensional or three-dimensional image data of the designed artificial bone, etc.).

The person-in-charge of the designing and manufacturing institution gains access to the URL of the artificial bone information managing system 1 through the designing and manufacturing institution side terminal device 400 (step S112).

Next, the authentication processing part 114 of the artificial bone information managing system 1 transmits input screen data for inputting the authentication ID and the password and the like (authentication information) to the designing and manufacturing institution side terminal device 400 (step S113).

Then, the designing and manufacturing institution side terminal device 400 receives and screen-inputs the input screen data. Upon reception of the authentication ID and the password inputted by the person-in-charge, the designing and manufacturing institution side terminal device 400 returns the received information back to the artificial bone information managing system 1 (step S114). The artificial bone information managing system 1 receives the returned information and the authentication processing part 114 thereof executes the authentication processing of the designing and manufacturing institution side terminal device 400 using the authentication database 127 (step S115).

If the result of the authentication processing shows rejection of authentication (authentication NG in step S115), the processing is terminated. In contrast, if the result of the authentication processing reveals acceptance of authentication (authentication OK in step S115), the artificial bone information acquiring part 112 of the artificial bone information managing system 1 becomes able to receive the artificial bone information from the designing and manufacturing institution side terminal device 400.

The designing and manufacturing institution side terminal device 400 is operated by the person-in-charge to transmit the artificial bone information to the artificial bone information managing system 1 (step S116).

The artificial bone information acquiring part 112 of the artificial bone information managing system 1 receives the artificial bone information transmitted from the designing and manufacturing institution side terminal device 400 and stores the artificial bone information in the associated storage location addresses of the artificial bone information database 126 (step S117). In the present embodiment, the order information database 125 and the artificial bone information database 126 are common and the storage location addresses of the order information are the same as the storage location addresses of the artificial bone information.

At the time when the designing and manufacturing institution side terminal device 400 transmits the artificial bone information to the artificial bone information managing system 1, a password may be assigned to the artificial bone information to thereby assure enhanced security. If necessary, the data of the artificial bone information may be encrypted or compressed. In these cases, the person-in-charge of the designing and manufacturing institution notifies the medical institution of the password or the like. The doctors of the medical institution notify the user or its family members of the password or the like. In the medical institution side terminal device 200 and the user side terminal device 300, the password or the like thus notified is used in receiving the artificial bone information from the artificial bone information managing system 1.

Thus, the artificial bone information transmitted from the designing and manufacturing institution side terminal device 400 is stored in the artificial bone information database 126. A notice of completion of storage of the artificial bone information is transmitted from the artificial bone information managing system 1 to the medical institution side terminal device 200 and the user side terminal device 300. The doctors of the medical institution and the user can understand from this notice that the storage of the artificial bone information has been completed.

The doctors of the medical institution gain access to the URL of the artificial bone information managing system 1 through the medical institution side terminal device 200 (step S118).

Next, the authentication processing part 114 of the artificial bone information managing system 1 transmits input screen data for inputting the authentication ID and the password and the like (authentication information) to the medical institution side terminal device 200 (step S119).

Subsequently, the medical institution side terminal device 200 receives and screen-inputs the input screen data. Upon reception of the authentication ID and the password inputted by the doctors, the medical institution side terminal device 200 returns the received information back to the artificial bone information managing system 1 (step S120). The artificial bone information managing system 1 receives the returned information and the authentication processing part 114 thereof executes the authentication processing of the medical institution side terminal device 200 using the authentication database 127 (step S121).

If the result of the authentication processing shows rejection of authentication (authentication NG in step S121), the processing is terminated. In contrast, if the result of the authentication processing reveals acceptance of authentication (authentication OK in step S121), the output processing part 113 of the artificial bone information managing system 1 becomes able to transmit the artificial bone information to the medical institution side terminal device 200.

The medical institution side terminal device 200 is operated by the doctors to transmit an extraction request of the artificial bone information to the artificial bone information managing system 1 (step S122).

Responsive to the extraction request, the output processing part 113 of the artificial bone information managing system 1 extracts the associated artificial bone information from the storage location addresses of the artificial bone information database 126 and transmits the extracted artificial bone information to the medical institution side terminal device 200 (step S123).

The medical institution side terminal device 200 stores the received artificial bone information in a storage device (storage means) not shown in the drawings. Then, the artificial bone information is outputted to, e.g., a display (display means) and a printer not shown in the drawings. That is to say, the artificial bone information is displayed on the display as an electronic image and also printed on a sheet of print paper so that the doctors of the medical institution can grasp the artificial bone to be used in surgery.

The patient or its family members gains access to the URL of the artificial bone information managing system 1 through the user side terminal device 300 (step S124).

Next, the authentication processing part 114 of the artificial bone information managing system 1 transmits input screen data for inputting the authentication ID and the password and the like (authentication information) to the user side terminal device 300 (step S125).

Subsequently, the user side terminal device 300 receives and screen-inputs the input screen data. Upon reception of the authentication ID and the password inputted by the patient or its family members, the user side terminal device 300 returns the received information back to the artificial bone information managing system 1 (step S126). The artificial bone information managing system 1 receives the returned information and the authentication processing part 114 thereof executes the authentication processing of the user side terminal device 300 using the authentication database 127 (step S127).

If the result of the authentication processing shows rejection of authentication (authentication NG in step S127), the processing is terminated. In contrast, if the result of the authentication processing reveals acceptance of authentication (authentication OK in step S127), the output processing part 113 of the artificial bone information managing system 1 becomes able to transmit the artificial bone information to the user side terminal device 300.

The user side terminal device 300 is operated by the patient or its family members to transmit an extraction request of the artificial bone information to the artificial bone information managing system 1 (step S128).

Responsive to the extraction request, the output processing part 113 of the artificial bone information managing system 1 extracts the associated artificial bone information from the storage location addresses of the artificial bone information database 126 and transmits the extracted artificial bone information to the user side terminal device 300 (step S129).

The user side terminal device 300 stores the received artificial bone information in a storage device (storage means) not shown in the drawings. Then, the artificial bone information is outputted to, e.g., a display (display means) and a printer not shown in the drawings. That is to say, the artificial bone information is displayed on the display as an electronic image and also printed on a sheet of print paper so that the user or its family members can grasp the artificial bone to be used in surgery.

As described above, the artificial bone information managing system 1 makes it possible to save the time, effort and cost involved in transporting the order information such as the diagnosis image data used in designing (manufacturing the artificial bone and also to remove any concern about the leakage of personal information which may occur when a third party person carries the diagnosis image data of a personal user managed by the medical institution.

Furthermore, it is possible to provide the doctors of the medical institution, the user who undergoes surgery of the artificial bone and its family members with an opportunity of, and a means for, minutely and clearly knowing prior information on the artificial bone surgically applied to the user. This makes it possible to efficiently provide the artificial bone information.

In the present invention, the authentication information (authentication ID and password) and the storage location addresses may be preliminarily transmitted to the person who agreed on a use contract.

In the present invention, one or both of the medical institution side terminal device 200 and the designing and manufacturing institution side terminal device 400 may be included in the elements of the artificial bone information managing system 1. Furthermore, the user side terminal device 300 may be included in the elements of the artificial bone information managing system 1.

Although it is assumed in the foregoing embodiment that the designing and manufacturing institution manages the artificial bone information managing system 1, the present invention is not limited thereto. Alternatively, the artificial bone information managing system 1 may be managed by, e.g., the medical institution or a third party other than the designing and manufacturing institution and the medical institution.

Although it is assumed in the foregoing embodiment that the artificial bone information managing system 1 exists between the medical institution side terminal device 200 and the designing and manufacturing institution side terminal device 400 and between the user side terminal device 300 and the designing and manufacturing institution side terminal device 400, the present invention is not limited thereto. The artificial bone information managing system 1 may be arranged in any position on the network 60. Additionally, in the present invention, the artificial bone information managing system 1 may also serve as the designing and manufacturing institution side terminal device 400 and as the medical institution side terminal device 200. In this regard, description will be made below with reference to second and third embodiments.

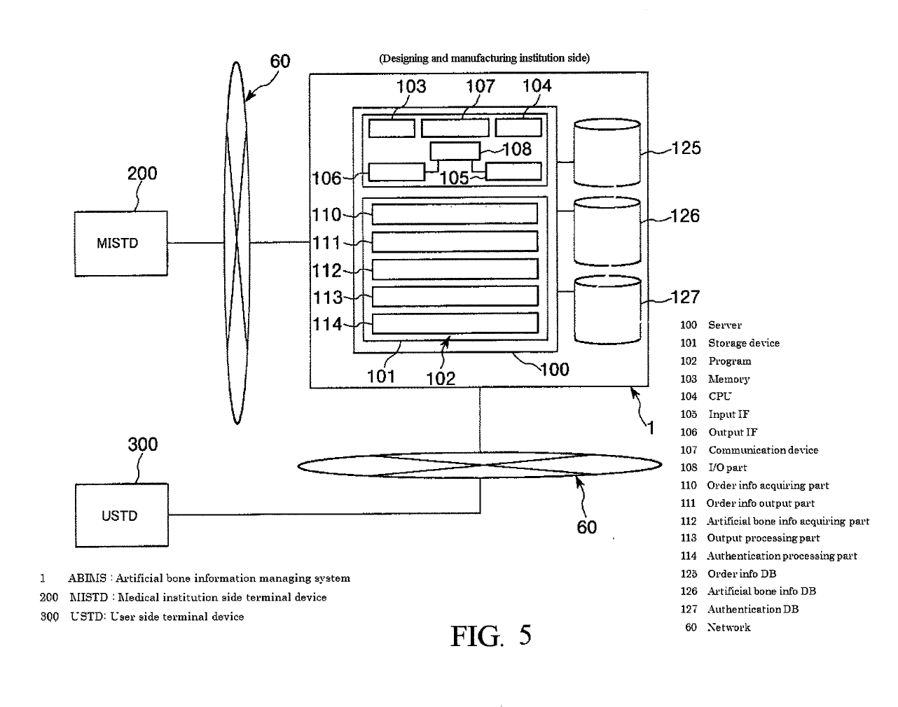

Next, a second embodiment of the present artificial bone information managing system will be described. FIG. 5 is a network configuration view (block diagram) showing a second embodiment of the present artificial bone information managing system.

Hereinafter, description of the artificial bone information managing system 1 of the second embodiment will be centered on the points differing from the first embodiment set forth above and no description will be given to the same matters.

The artificial bone information managing system 1 of the second embodiment is the same as the first embodiment described above, except that it also serves as the designing and manufacturing institution side terminal device.

As shown in FIG. 5, the artificial bone information managing system 1 of the second embodiment is installed in the designing and manufacturing institution and serves as the designing and manufacturing institution side terminal device.

Description will be briefly made on the respective function parts that are configured and maintained by the artificial bone information managing system 1 according to the program 102.

The order information acquiring part (order information acquiring means) 110 is a function part (means) that receives the order information including the diagnosis image data of the user required in designing the artificial bone from the medical institution side terminal device 200 through the network 60 and then stores the order information in the order information database 125.

The order information output part (order information output means) 111 is a function part (means) that accepts an extraction request of the order information, extracts the associated order information from the order information database 125 in response to the extraction request and outputs the extracted order information.

The artificial bone information acquiring part (artificial bone information acquiring means) 112 is a function part (means) by which the artificial bone information on the artificial bone designed by the designing and manufacturing institution according to the order information is stored in the artificial bone information database 126.

The output processing part (output processing means) 113 is a function part (means) that accepts an extraction request of the artificial bone information through the network 60, extracts the associated artificial bone information from the artificial bone information database 126 in response to the extraction request and transmits the extracted artificial bone information to the medical institution side terminal device 200 or the user side terminal device 300 through the network 60.

The artificial bone information managing system 1 of the second embodiment provides the same effects as those attained by the first embodiment.

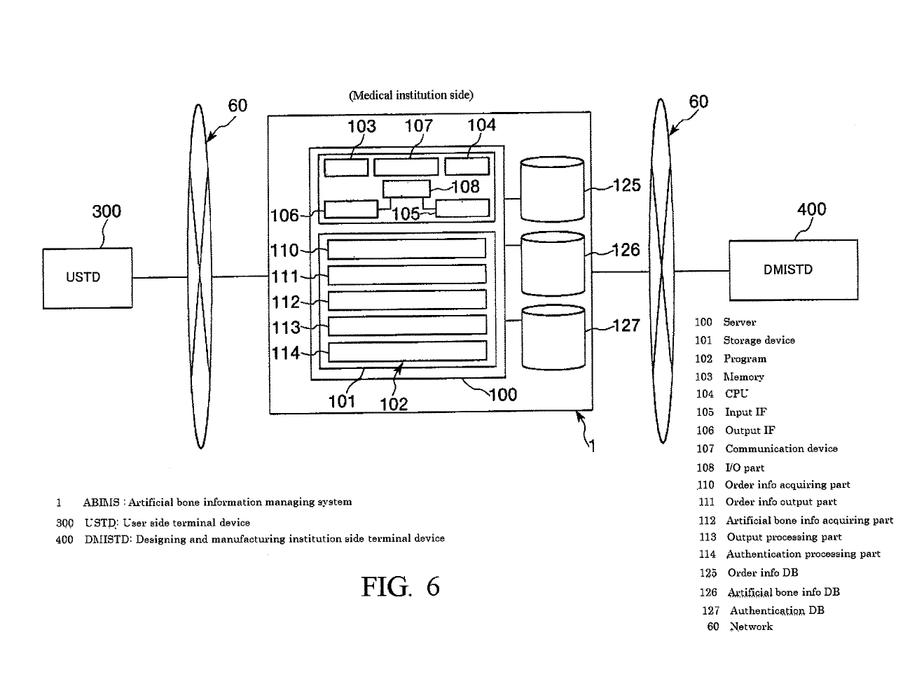

Next, a third embodiment of the present artificial bone information managing system will be described. FIG. 6 is a network configuration view (block diagram) showing a third embodiment of the present artificial bone information managing system.

Hereinafter, description of the artificial bone information managing system 1 of the third embodiment will be centered on the points differing from the first embodiment set forth above and no description will be given to the same matters.

The artificial bone information managing system 1 of the third embodiment is the same as the first embodiment described above, except that it serves as the medical institution side terminal device.

As shown in FIG. 6, the artificial bone information managing system 1 of the third embodiment is installed in the medical institution and serves as the medical institution side terminal device.

Description will be briefly made on the respective function parts that are configured and maintained by the artificial bone information managing system 1 according to the program 102.

The order information acquiring part (order information acquiring means) 110 is a function part (means) by which the order information including the diagnosis image data of the user required in designing the artificial bone is stored in the order information database 125.

The order information output part (order information output means) 111 is a function part (means) that accepts an extraction request of the order information through the network 60, extracts the associated order information from the order information database 125 in response to the extraction request and transmits the extracted order information to the designing and manufacturing institution side terminal device 400 through the network 60.

The artificial bone information acquiring part (artificial bone information acquiring means) 112 is a function part (means) that receives the artificial bone information on the artificial bone designed by the designing and manufacturing institution according to the order information from the designing and manufacturing institution side terminal device 400 and then stores the received artificial bone information in the artificial bone information database 126.

The output processing part (output processing means) 113 is a function part (means) that accepts an extraction request of the artificial bone information, extracts the associated artificial bone information from the artificial bone information database 126 in response to the extraction request and outputs the extracted artificial bone information or transmits the extracted artificial bone information to the user side terminal device 300 through the network 60.

In the artificial bone information managing system 1 of the third embodiment, the artificial bone information is outputted to various kinds of external devices such as a display (display means) and a printer. That is to say, the artificial bone information is displayed on the display as an electronic image and also printed on a sheet of print paper so that the doctors of the medical institution can see the artificial bone information.

The artificial bone information managing system 1 of the third embodiment provides the same effects as those attained by the first embodiment.

While the artificial bone information managing system of the present invention has been described with reference to the embodiments illustrated in the drawings, the present invention is not limited thereto. It may be possible to replace each part of the system with a part of an arbitrary configuration having the same function. It may also be possible to add other arbitrary components or steps to the invention.

Moreover, the present invention may be a combination of two or more arbitrary configurations (features) employed in the respective embodiments set forth above.

Finally, it is also to be understood that the present disclosure relates to the subject matter contained in Japanese Patent Application No. 2006-324955 filed on Nov. 30, 2006 which is expressly incorporated herein by reference in its entirety.

1. An artificial bone information managing system that performs the management of information on an artificial bone which is ordered to a designing and manufacturing institution by a medical institution and which is designed and manufactured on a user-by-user basis by the designing and manufacturing institution, the system comprising:
an order information acquirer that receives the order information including the diagnosis image data of the user required in designing the artificial bone from a medical institution side terminal device through a network and then stores the order information in an order information database;
an order information outputter that receives an extraction request of the order information through the network, extracts the associated order information from the order information database in response to the order information extraction request and transmits the extracted order information to the designing and manufacturing institution side terminal device through the network;
an artificial bone information acquirer that receives the artificial bone information on the artificial bone designed by the designing and manufacturing institution according to the order information from the designing and manufacturing institution side terminal device through the network and stores the artificial bone information in an artificial bone information database; and
an output processor that receives an extraction request of artificial bone information through the network, extracts the associated artificial bone information from the artificial bone information database in response to the artificial bone information extraction request and transmits the extracted artificial bone information to the medical institution side terminal device or the user side terminal device through the network.

2. The artificial bone information managing system as claimed in claim 1, wherein the order information is deleted from the order information database after the order information has been transmitted to the designing and manufacturing institution side terminal device by the order information means outputter.

3. The artificial bone information managing system as claimed in claim 1, further comprising an authentication processor that transmits storage location addresses of the order information allotted in the order information database and authentication information specific to the designing and manufacturing institution required in authenticating the designing and manufacturing institution to the designing and manufacturing institution side terminal device before the order information outputter receives the extraction request of the order information from the designing and manufacturing institution side terminal device, wherein the authentication processor performs verification of the authentication information to thereby authenticate the designing and manufacturing institution.

4. The artificial bone information managing system as claimed in claim 3, wherein the system is configured so that at least one of the storage location addresses of the order information and the authentication information to be transmitted to the designing and manufacturing institution side terminal device is changed upon each transmission thereof.

5. The artificial bone information managing system as claimed in claim 3, wherein the order information database and the artificial bone information database are consolidated into a common one, and the storage location addresses of the order information are the same as the storage location addresses of the artificial bone information.

6. An artificial bone information managing system that performs the management of information on an artificial bone which is ordered to a designing and manufacturing institution by a medical institution and which is designed and manufactured on a user-by-user basis by the designing and manufacturing institution, the system comprising:
an order information acquirer that receives the order information including the diagnosis image data of the user required in designing the artificial bone from a medical institution side terminal device through a network and then stores the order information in an order information database;
an order information outputter that receives an extraction request of the order information, extracts the associated order information from the order information database in response to the order information extraction request and outputs the extracted order information;
an artificial bone information acquirer that stores the artificial bone information on the artificial bone designed by the designing and manufacturing institution according to the order information in an artificial bone information database; and
an output processor that receives an extraction request of artificial bone information through the network, extracts the associated artificial bone information from the artificial bone information database in response to the artificial bone information extraction request and transmits the extracted artificial bone information to the medical institution side terminal device or the user side terminal device through the network,
wherein all of the order information acquirer, the order information outputter, the artificial bone information acquirer, and the output processor are located at the side of the designing and manufacturing institution.

7. The artificial bone information managing system as claimed in claim 6, wherein the system is configured so that the order information is deleted from the order information database after the order information has been transmitted by the order information outputter.

8. The artificial bone information managing system as claimed in claim 1, wherein the output processor is configured so that when the artificial bone information extraction request from the medical institution side terminal device is received through the network, the output processor extracts the associated artificial bone information from the artificial bone information database in response to the information extraction request and transmits the extracted artificial bone information to the medical institution side terminal device, and wherein the output processor is also configured so that when the artificial bone information extraction request from the user side terminal device is received through the network, the output processor extracts the associated artificial bone information from the artificial bone information database in response to the artificial bone information extraction request and transmits the extracted artificial bone information to the user side terminal device.

9. The artificial bone information managing system as claimed in claim 1, further comprising an authentication processor that transmits storage location addresses of the order information allotted in the order information database and authentication information specific to the medical institution required in authenticating the medical institution to the medical institution side terminal device before the order information acquirer receives the order information from the medical institution side terminal device, wherein the authentication processor performs verification of the authentication information to thereby authenticate the medical institution.

10. The artificial bone information managing system as claimed in claim 9, wherein the system is configured so that at least one of the storage location addresses of the order information and the authentication information to be transmitted to the medical institution side terminal device is changed upon each transmission thereof.

11. The artificial bone information managing system as claimed in claim 9, wherein the order information database and the artificial bone information database are consolidated into a common one, and the storage location addresses of the order information are the same as the storage location addresses of the artificial bone information.

12. An artificial bone information managing system that performs the management of information on an artificial bone which is ordered to a designing and manufacturing institution by a medical institution and which is designed and manufactured on a user-by-user basis by the designing and manufacturing institution, the system comprising:
an order information acquirer that stores the order information including the diagnosis image data of the user required in designing the artificial bone in an order information database;
an order information outputter that receives an extraction request of the order information through the network, extracts the associated order information from the order information database in response to the order information extraction request and transmits the extracted order information to the designing and manufacturing institution side terminal device through the network;
an artificial bone information acquirer that receives the artificial bone information on the artificial bone designed by the designing and manufacturing institution according to the order information from the designing and manufacturing institution side terminal device through the network and stores the artificial bone information in an artificial bone information database; and
an output processor that receives an extraction request of artificial bone information through the network, extracts the associated artificial bone information from the artificial bone information database in response to the artificial bone information extraction request and transmits the extracted artificial bone information to the user side terminal device through the network,
wherein all of the order information acquirer, the order information outputter, the artificial bone information acquirer, and the output processor are located at the side of the medical institution.

13. The artificial bone information managing system as claimed in claim 12, wherein the system is configured so that the order information is deleted from the order information database after the order information has been transmitted to the designing and manufacturing institution side terminal device by the order information outputter.

14. The artificial bone information managing system as claimed in claim 12, wherein the output processor is configured so that when the artificial bone information extraction request from the user side terminal device is received through the network, the output processor extracts the associated artificial bone information from the artificial bone information database in response to the artificial bone information extraction request and transmits the extracted artificial bone information to the user side terminal device through the network.

15. The artificial bone information managing system as claimed in claim 12, further comprising an authentication processor that transmits storage location addresses of the order information allotted in the order information database and authentication information specific to the designing and manufacturing institution required in authenticating the designing and manufacturing institution to the designing and manufacturing institution side terminal device before the order information acquirer receives the order information from the designing and manufacturing institution side terminal device, wherein the authentication processor performs verification of the authentication information to thereby authenticate the medical institution.

16. The artificial bone information managing system as claimed in claim 15, wherein the system is configured so that at least one of the storage location addresses of the order information and the authentication information to be transmitted to the designing and manufacturing institution side terminal device is changed upon each transmission thereof.

17. The artificial bone information managing system as claimed in claim 15, wherein the order information database and the artificial bone information database are consolidated into a common one, and the storage location addresses of the order information are the same as the storage location addresses of the artificial bone information.

18. The artificial bone information managing system as claimed in claim 1, wherein the diagnosis image data of the user includes a plurality of tomographic image data which indicate an installation position of the artificial bone of the user.

19. The artificial bone information managing system as claimed in claim 1, wherein the order information include one of the information on an electronic medical chart of the user and the information on a material of the artificial bone.

20. The artificial bone information managing system as claimed in claim 1, wherein the artificial bone information includes at least one of the two-dimensional image data and three-dimensional image data of the designed artificial bone.

21. The artificial bone information managing system as claimed in claim 1, wherein the artificial bone information includes at least one of a size, a material, a color, a function and a past use record of the designed artificial bone.
